(19) United States
(12) Patent Application Publication
(10) Pub. No.: US 2021/0313018 A1
(43) Pub. Date: Oct. 7, 2021

(54) PATIENT ASSESSMENT SUPPORT DEVICE, PATIENT ASSESSMENT SUPPORT METHOD, AND RECORDING MEDIUM

(71) Applicant: NEC CORPORATION, Tokyo (JP)

(72) Inventors: Yuan LUO, Tokyo (JP); Masahiro KUBO, Tokyo (JP); Toshinori HOSOI, Tokyo (JP); Yuki KOSAKA, Tokyo (JP); Masahiro HAYASHITANI, Tokyo (JP); Yuji OHNO, Tokyo (JP); Yutaka UNO, Tokyo (JP); Eiji YUMOTO, Tokyo (JP); Shigemi KITAHARA, Tokyo (JP)

(73) Assignee: NEC CORPORATION, Tokyo (JP)

(21) Appl. No.: 17/255,346

(22) PCT Filed: Jun. 27, 2019
(86) PCT No.: PCT/JP2019/025631

§ 371 Date: Dec. 22, 2020

(30) Foreign Application Priority Data
Jun. 29, 2018 (JP) 2018-124478

(51) Int. Cl. G16H 10/20; G16H 10/60; G16H 50/70

(52) U.S. Cl. CPC G16H 10/20; G16H 50/70; G16H 10/60

(57) ABSTRACT

A patient assessment support device includes an output unit which outputs estimated support information estimated based on question information relating to nursing care for a patient when a record of known support information relating to the patient is determined as being absent.

### TECHNICAL FIELD

The present invention relates to a patient assessment support device, a patient assessment support method, and a recording medium.

### BACKGROUND ART

Duties of nurses cover almost all hospital ward duties. Optimization of the duties of nurses is essential in order to optimize the overall hospital ward duties. In executing such optimization, patient assessment is considered as the most important point for the efficiency of nurse's duties. However, at present, there are large individual differences in patient assessment, depending on the duration of service and experience of nurses. In particular, junior nurses lack experience, and thus the level of efficiency in their work related to patient assessment is low, which may result in errors in their work. The term patient assessment here means an evaluation or assessment of the condition of a patient.

As a related technique, Patent Document 1 discloses a technique capable of supporting cooperation in patient care. For example, paragraph 0106 of Patent Document 1 describes that “the report display processing unit 405 executes branch processing upon the patient state information screen being selected, . . . the report display processing unit 405 advances the process to Step S209 if ‘Report detail’ is selected”. Also, paragraph 0107 of Patent Document 1 describes that “the report display processing unit 405 causes the user terminal 20 to display the details of the observation result information of each classification item as shown in FIG. 11”. Furthermore, FIG. 11 of Patent Document 1 shows a display screen of report details of patient observation information.

Moreover, Patent Document 2 discloses a technique of a medical support system which reduces waiting time by provisionally determining the ranks of patients on the basis of the states of patients visiting a hospital to receive chemotherapy, and which improves the reliability of case report forms (CRF).

### PRIOR ART DOCUMENTS

### Patent Documents

[Patent Document 1] Japanese Unexamined Patent Application, First Publication No. 2016-212925

[Patent Document 2] Japanese Unexamined Patent Application, First Publication No. 2015-170159

### SUMMARY OF THE INVENTION

### Problem to be Solved by the Invention

There is a demand for a technique capable of promptly supporting appropriate nursing care for a patient even in a situation where the nurse has limited experience in patient assessment.

An example of an object of the present invention is to provide a patient assessment support device, a patient assessment support method, and a recording medium capable of solving the problems mentioned above.

### Means for Solving the Problem

According to a first example aspect of the present invention, a patient assessment support device includes: an output unit which outputs estimated support information estimated based on question information relating to nursing care for a patient when a record of known support information relating to the patient is determined as being absent.

According to a second example aspect of the present invention, a patient assessment support method includes: outputting estimated support information estimated based on question information relating to nursing care for a patient when a record of known support information relating to the patient is determined as being absent.

According to a third example aspect of the present invention, a recording medium causes a computer to execute: outputting estimated support information estimated based on question information relating to nursing care for a patient when a record of known support information relating to the patient is determined as being absent.

### Effect of the Invention

According to an example embodiment of the invention, it is possible to promptly support appropriate nursing care for a patient even in a situation where a nurse has limited experience in patient assessment.

### BRIEF DESCRIPTION OF THE DRAWINGS

FIG. 1 is a block diagram showing a configuration of a patient assessment support system according to a first example embodiment.

FIG. 2 is a hardware configuration diagram of a patient assessment support device shown in FIG. 1.

FIG. 3 is a functional block diagram of the patient assessment support device shown in FIG. 1.

FIG. 4 is a first diagram showing a processing flow of the patient assessment support device shown in FIG. 1.

FIG. 5 is a second diagram showing a processing flow of the patient assessment support device shown in FIG. 1.

FIG. 6 is a first diagram showing an example of recorded information of the patient assessment support device shown in FIG. 1.

FIG. 7 is a second diagram showing an example of recorded information of the patient assessment support device shown in FIG. 1.

FIG. 8 is a diagram showing a configuration of a patient assessment support device according to a second example embodiment.

### EXAMPLE EMBODIMENTS

Hereinafter, a patient assessment support device according to example embodiments of the present invention will be described, with reference to the drawings.

FIG. 1 is a block diagram showing a configuration of a patient assessment support system according to a first example embodiment. As shown in FIG. 1, a patient assessment support system 100 includes at least a patient assessment support device 1 and a database 2.

The patient assessment support device 1 and the database 2 are communicatively connected to each other. The database 2 records information for supporting patient care performed by nurses, doctors and so forth. The patient assessment support device 1 generates estimated support information on the basis of the information recorded in the database 2. A device of an output destination such as a mobile terminal 3 is communicatively connected to the patient assessment support device 1. The patient assessment support device 1 outputs known support information and estimated support information to the mobile terminal 3. The mobile terminal 3 is used by a nurse or a doctor. The nurse or the doctor may carry an output destination device other than the mobile terminal 3. Furthermore, the patient assessment support device 1 may output known support information or estimated support information to such a device other than the mobile terminal 3. The term known support information here may be known information (information already recorded in a database or the like) related to patient support. The estimated support information may mean estimated information regarding patient support.

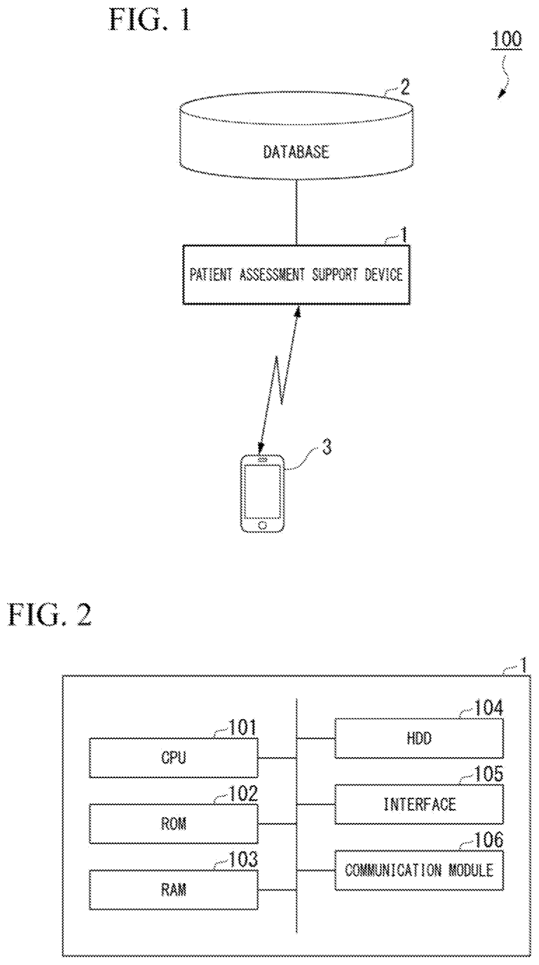

FIG. 2 is a hardware configuration diagram of the patient assessment support device 1.

The patient assessment support device 1 is a computer, and, as shown in FIG. 2, includes hardware such as a CPU (Central Processing Unit) 101, a ROM (Read Only Memory) 102, a RAM (Random Access Memory) 103, a HDD (Hard Disk Drive) 104, an interface 105, and a communication module 106. The patient assessment support device 1 may be configured with a single computer as shown in FIG. 2, or may be configured with a plurality of computers connected communicatively.

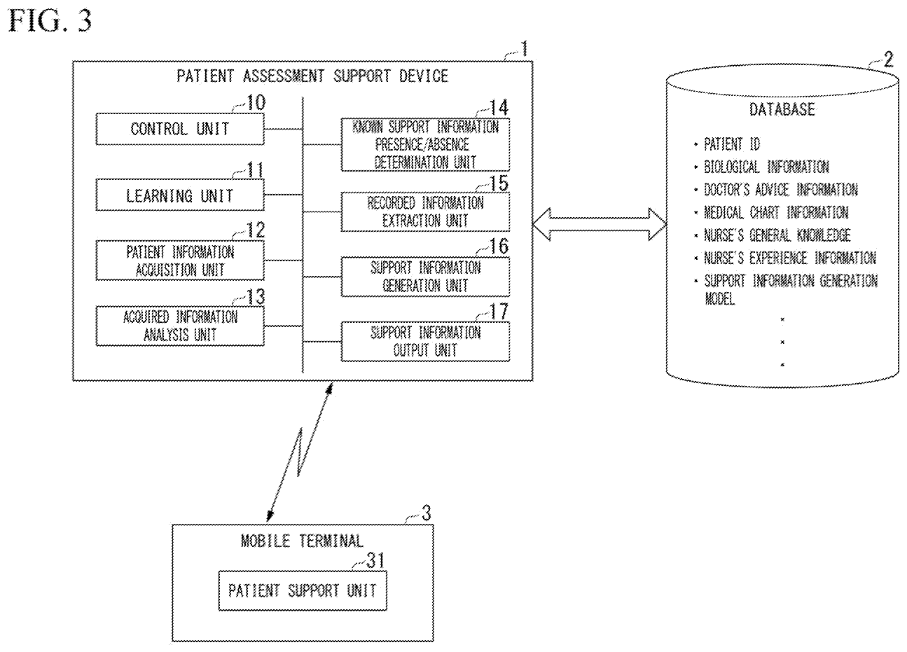

FIG. 3 is a functional block diagram of the patient assessment support device 1 and the mobile terminal 3.

Upon activation, the patient assessment support device 1 executes a patient assessment support program stored in the device itself (the patient assessment support device 1). Thereby, the patient assessment support device 1 is enabled to execute the function of a control unit 10, a learning unit 11, a patient information acquisition unit (an acquisition unit) 12, an acquired information analysis unit 13, a known support information presence/absence determination unit (a determination unit) 14, a recorded information extraction unit 15, a support information generation unit (a generation unit) 16, and a support information output unit (an output unit) 17.

The control unit 10 controls the patient assessment support device 1.

The learning unit 11 generates learning data of a support information generation model, using information recorded in the database and so forth.

The patient information acquisition unit 12 acquires patient identification information for identifying a patient and question information for the patient.

The acquired information analysis unit 13 analyzes the question information.

The known support information presence/absence determination unit 14 determines whether or not known support information for the patient is present, on the basis of the patient identification information.

The recorded information extraction unit 15 extracts from the database 2, information used for known support information or estimated support information.

If known support information is determined as being absent, the support information generation unit 16 generates estimated support information, on the basis of the question information included in patient information.

The support information output unit 17 outputs known support information or estimated support information to a predetermined mobile terminal 3 which has transmitted the patient information.

With such a configuration, even in a situation where a nurse has limited experience in patient assessment, the patient assessment support device 1 outputs known support information or estimated support information to the mobile terminal 3 of the nurse and so forth and thereby supports appropriate nursing care for the patient.

The mobile terminal 3 includes a patient support unit 31 by a patient support application program being activated.

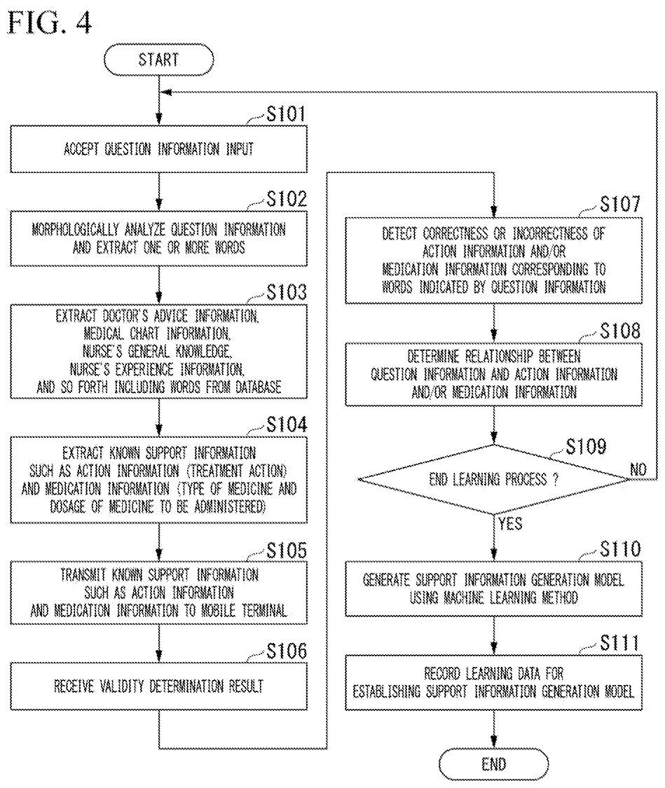

FIG. 4 is a first diagram showing a processing flow of the patient assessment support device 1.

First, a process in which the patient assessment support device 1 generates learning data will be described.

The learning unit 11 of the patient assessment support device 1 accepts an input of question information predicted in advance, and generates learning data of a support information generation model for generating support information corresponding to the question information. Specifically, the learning unit 11 accepts an input of question information (Step S101). For example, a user such as a doctor or a nurse may transmit the question information to the patient assessment support device 1 using a mobile terminal 3 or the like, and the learning unit 11 of the patient assessment support device 1 may accept the question information.

The learning unit 11 performs morphological analysis on the question information and extracts one or a plurality of words (Step S102). The learning unit 11 extracts from the database 2 doctor's advice information, medical record information (medical chart information), nurse's general knowledge, nurse's experience information, and so forth which include these words (Step S103). Among the items included in the doctor's advice information, the medical record information, the nurse's general knowledge, the nurse's experience information, and so forth, the learning unit 11 extracts known support information such as action information (treatment action), medication information (type of medicine, dosage of medicine to be administered), and so forth (Step S104). The learning unit 11 transmits the extracted known support information such as the action information and the medication information to the mobile terminal 3 (Step S105).

The doctor, the nurse or the like using the mobile terminal 3 determines the validity of the answer to the question information regarding the action information and the medication information received from the patient assessment support device 1 in response to the transmitted question information. The doctor, the nurse, or the like inputs into the mobile terminal 3 a determined result (correct or incorrect) of the validity of the answer.

The patient assessment support device 1 receives the validity determined result from the mobile terminal 3 (Step S106). Based on the reception of the validity determination result, the learning unit 11 detects the correctness or incorrectness of the action information and the medication information corresponding to the words indicated by the question information (Step S107). Based on the action information and the medication information determined as being correct among the extracted action information and medication information corresponding to the question information, the learning unit 11 determines the relationship between the question information, and the action information and the medication information (Step S108). The learning unit 11 determines whether or not to end the learning process (Step S109). The learning unit 11 does not end the learning process if repetitions of input of various question information, extraction of action information and medication information, and validity determination result input corresponding thereto are insufficient. The learning unit 11 repeats input of various question information, extraction of action information and medication information, and input of validity determination result corresponding to the extracted information, to identify highly valid action information and medication information which correspond to the question information.

Then, the learning unit 11 generates a support information generation model for extracting highly valid action information and medication information for the question information, using a machine learning method (Step S110). The learning unit 11 records in the database 2 learning data and so forth for establishing the support information generation model (Step S111). The learning unit 11 may generate a support information generation model again at a timing at which the biological information, the doctor's advice information, and the medical record information recorded in the database 2 increases.

The learning unit 11 may, by means of another method, generate the support information generation model for identifying action information and medication information for question information. For example, question information may include biological information including patient's condition and so forth. The learning unit 11 may extract corresponding action information and medication information using such question information, and may generate a support information generation model upon receiving an input of the result of the validation thereof from a nurse or the like. Biological information is information of body temperature, heart rate, and other biological or condition information.

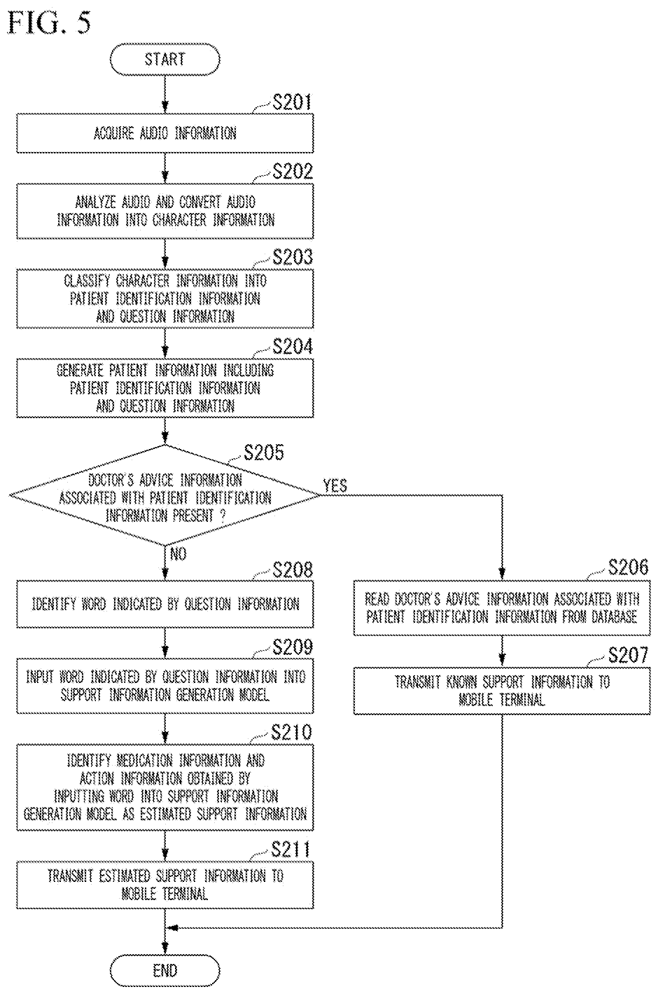

FIG. 5 is a second diagram showing a processing flow of the patient assessment support device 1.

The patient assessment support device 1 having generated a support information generation model can provide support corresponding to an actual patient assessment (evaluation, assessment). Specifically, when a nurse is to actually perform patient care, the nurse uses the patient assessment support device 1 in a case where their knowledge on patient's case is insufficient or in a situation where the nurse is unable to return to the nurse station and confirm information materials such as medical records. The nurse activates the patient support application program recorded on the mobile terminal 3. As a result, the mobile terminal 3 becomes able to execute functions of the patient support unit 31.

The patient support unit 31 displays on a monitor of the mobile terminal 3, a screen instructing to input patient identification information and question information. Also, the patient support unit 31 activates a microphone provided in the mobile terminal 3. The nurse using the mobile terminal 3 states at least one of patient identification information such as patient ID, full name, bed number, and room number and question information, into the microphone of the mobile terminal 3. The question information may include information indicating the patient's condition (body temperature, heart rate, and information indicating other conditions). The microphone transmits the voice information including the patient identification information and the question information to the patient assessment support device 1. The patient information acquisition unit 12 of the patient assessment support device 1 acquires the received voice information (Step S201). The patient information acquisition unit 12 outputs the voice information to the acquired information analysis unit 13.

The acquired information analysis unit 13 acquires the voice information and performs a voice analysis thereon, and converts the voice information into character information (Step S202). The acquired information analysis unit 13 analyzes the character sequence of the character information corresponding to the voice information, and classifies the character information into patient identification information and question information (Step S203). The acquired information analysis unit 13 may perform a morphological analysis on the character information and may extract words of the patient identification information and the question information. The acquired information analysis unit 13 generates post-classification patient information which includes the patient identification information and the question information (Step S204). Commonly known techniques may be used for the methods for the voice analysis, the character sequence analysis of character information, the classification of patient identification information and question information, and the morphological analysis performed by the acquired information analysis unit 13. The acquired information analysis unit 13 outputs the patient identification information (at least one of the patient ID, the full name, bed number and the room number) to the known support information presence/absence determination unit 14.

Upon acquiring the patient identification information, the known support information presence/absence determination unit 14 determines whether or not any doctor's advice information associated with the patient identification information is present (Step S205). The doctor's advice information is information including, for example, type of medicine and dosage of medicine to be administered according to patient's condition, and treatment action and so forth. If doctor's advice information is recorded in the database 2, the known support information presence/absence determination unit 14 outputs the patient identification information to the recorded information extraction unit 15. The recorded information extraction unit 15 reads the doctor's advice information associated with the patient identification information from the database 2 (Step S206). The recorded information extraction unit 15 outputs known support information which includes doctor's advice information to the support information output unit 17. The support information output unit 17 transmits the known support information including the doctor's advice information to the mobile terminal 3 (Step S207).

The patient support unit 31 of the mobile terminal 3 outputs the known support information to the monitor. The patient support unit 31 of the mobile terminal 3 may output the character sequence of the known support information by voice from a speaker. As a result, the user of the mobile terminal 3 such as a nurse can confirm the medication type, the medication dosage, the treatment action, and so forth from the doctor's advice information indicated by the known support information, and perform an appropriate treatment indicated by a doctor.

If doctor's advice information being known support information is determined as being absent in Step S205, the known support information presence/absence determination unit 14 instructs the support information generation unit 16 to generate estimated support information. The support information generation unit 16 acquires question information from the acquired information analysis unit 13. The support information generation unit 16 identifies words indicated by the question information (Step S208). The words indicated by the question information are, for example, words which indicate patient's disease name, and patient's condition (body temperature, heart rate, blood pressure, incidence of disease, and information indicating other conditions).

The support information generation unit 16 inputs the words indicated by the question information into the support information generation model (Step S209). The support information generation model is a model to which a convolutional neural network or the like is applied, and it outputs medication information (type of medicine, dosage of medicine to be administered) and action information (treatment action) according to one or more words indicated by question information. The support information generation unit 16 identifies, as estimated support information, the medication information and the action information obtained by inputting the words into the support information generation model (Step S210). The support information generation unit 16 outputs the estimated support information including the medication information and the action information, to the support information output unit 17. The support information output unit 17 transmits the estimated support information to the mobile terminal 3 (Step S211). The patient support unit 31 of the mobile terminal 3 outputs the estimated support information to the monitor. The patient support unit 31 of the mobile terminal 3 may output the character sequence which indicates the estimated support information by voice from a speaker.

According to the above processing, the user such as a nurse who uses the mobile terminal 3 by connecting it to the patient assessment support device 1 can acquire known support information or estimation support information according to question information about a patient, and can perform nursing treatment actions based on this support information.

Moreover, according to the above processing, even in a case where the database 2 does not have known support information such as doctor's advice information regarding a specific patient stored therein, nursing treatment actions of highly skilled nurses can be recognized by means of the support information generation model. As a result, the user such as a nurse who uses this system can promptly perform a correct nursing treatment. The reason for this is that this system can learn general knowledge, past experiences of plurality of experienced nurses (nurse's general knowledge, nurse's experience information) and information of individual patients, can determine the status of patients at the site where nursing treatments are actually performed, and can propose corresponding actions.

Next, an operation of the example embodiment of the present invention will be described, using a specific example.

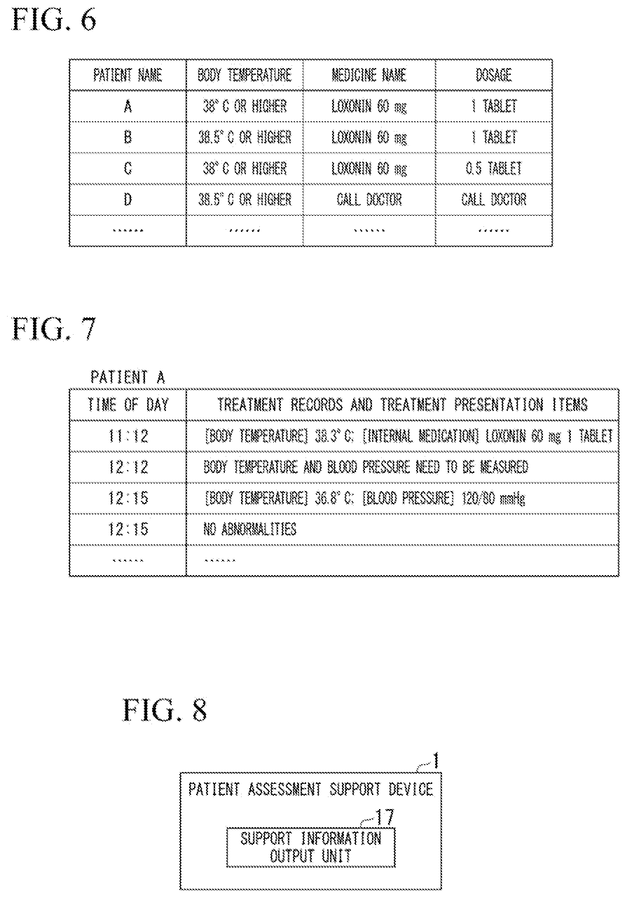

FIG. 6 is a first diagram showing an example of recorded information of the patient assessment support device 1.

As shown in FIG. 6, the condition and the dosage of medicine to be administered differs from patient to patient. A nurse can receive and confirm such information on the mobile terminal 3 carried by the nurse. Therefore, for example, the user such as a nurse can promptly confirm doctor's instructions or nursing treatment action information of other experienced nurses in a real-time fashion at the site where nursing treatments are actually performed, without having to return to the nurse station.

The patient information acquisition unit 12 and the acquired information analysis unit 13 of the patient assessment support device 1 may, in a database, record voice information received from the mobile terminal 3 and/or the result of analyzing the voice information. As a result, the patient assessment support device 1 can record the status of a treatment performed by a nurse during a nursing task.

FIG. 7 is a second diagram showing an example of recorded information of the patient assessment support device 1.

The patient assessment support device 1 can, in the processing described above, record a treatment action concerning a specific patient (FIG. 7). For example, in the above processing, the acquired information analysis unit 13 of the patient assessment support device 1 analyzes the voice information and converts it into character information, and records in the database 2 the patient identification information and other information included in the character information in association with the time of receiving the voice information. For example, as shown in FIG. 4, for a patient A, it is recorded that a nurse detected fever at 11:12 and administered one 60 mg tablet of Loxonin (name of medicine).

The support information generation unit 16 of the patient assessment support device 1 may learn past nursing treatment actions on the basis of a past record based on voice information and the like regarding a patient in the past. In such a case, the support information generation unit 16 may, if information on the fever and the completion of administration of medicine has been newly recorded for the patient A, automatically generate a subsequent treatment action and may output it as support information. For example, the support information generation unit 16 determines that it is necessary to confirm the body temperature and the blood pressure of the patient A at 12:12, one hour after taking Loxonin, and generates estimated support information which includes nursing treatment action information prompting to measure body temperature and blood pressure. The support information output unit 17 may transmit the estimated support information to the mobile terminal 3. As a result, the nurse recognizes that patient A needs to have body temperature and blood pressure measured. Moreover, the nurse voice-inputs the completion of the body temperature measurement and the blood pressure measurement for the patient A, using the mobile terminal 3. As a result, the acquired information analysis unit 13 records information which indicates that the measurement of body temperature and the measurement of blood pressure have been completed for the patient A in a manner similar to the processing described above. The support information generation unit 16 generates support information again after information is newly recorded. For example, the support information generation unit 16 may determine, from the past records of the patient A, as “no abnormality” and may generate estimated support information indicating this information. The nurse can confirm “no abnormality” displayed on the mobile terminal 3.

FIG. 8 is a diagram showing a configuration of the patient assessment support device 1 according to a second example embodiment.

If it is determined, based on the acquired patient identification information, that there is no record of known support information for a patient, the support information output unit (the output unit) 17 outputs estimated support information estimated based on input question information.

Each device described above has a built-in computer system. The process of each processing described above is stored in a computer-readable recording medium in a form of a program, and the processing mentioned above is performed by a computer reading and executing the program. Here, the computer-readable recording medium refers to a magnetic disk, a magnetic optical disk, a CD-ROM, a DVD-ROM, a semiconductor memory, or the like. Moreover, the computer program may be distributed to a computer via a communication line, and the computer having received the distributed program may execute the program.

Also, this program may be a program for realizing some of the functions described above. Furthermore, the program may be a so-called difference file (a difference program) which can realize the functions described above in combination with a program already recorded in the computer system.

This application is based upon and claims the benefit of priority from Japanese patent application No. 2018-124478, filed Jun. 29, 2018, the disclosure of which is incorporated herein in its entirety by reference.

### INDUSTRIAL APPLICABILITY

The present invention may be applied to a patient assessment support device, a patient assessment support method, and a recording medium.

### REFERENCE SYMBOLS

1 Patient assessment support device

### 2 Database

3 Mobile terminal
10 Control unit
11 Learning unit
12 Patient information acquisition unit (acquisition unit)
13 Acquired information analysis unit
14 Known support information presence/absence determination unit (determination unit)
15 Recorded information extraction unit
16 Support information generation unit (generation unit)
17 Support information output unit (output unit)
31 Patient support unit

1. A patient assessment support device comprising:
at least one memory configured to store instructions; and
at least one processor configured to execute the instructions to:
acquire patient identification information for identifying a patient, and question information relating to nursing care for the patient
identify the patient based on the patient identification information;
determine whether or not known support information for the identified patient is present;
generate estimated support information based on the question information in response to determination that the known support information for the identified patient is absent and
output the estimated support information.

2. (canceled)

3. The patient assessment support device according to claim 1,
wherein the known support information is information stored in association with the patient identification information, and includes at least one of patient biological information relating to a living body of the patient, doctor's instruction information indicating a doctor's instruction relating to the patient, and medical record information indicating a medical record relating to the patient, and
the processor is configured to execute the instructions to output at least one of the patient biological information, the doctor's instruction information, and the medical record information in response to determination that the known support information is present.

4. The patient assessment support device according to claim 1, wherein the processor is configured to execute the instructions to estimate the estimated support information corresponding to the question information based on a support information generation model in response to determination that the known support information is determined as being absent.

5. The patient assessment support device according to claim 1 the processor is configured to execute the instructions to:
generate learning data of a support information generation model for generating the estimated support information corresponding to the question information.

6. A patient assessment support method comprising:
acquiring patient identification information for identifying a patient, and question information relating to nursing care for the patient
identifying the patient based on the patient identification information;
determining whether or not known support information for the identified patient is present;
generating estimated support information based on the question information in response to determination that the known support information for the identified patient is absent and
outputting the estimated support information.

7. A non-transitory computer readable recording medium storing a program which causes a computer to execute:
acquiring patient identification information for identifying a patient, and question information relating to nursing care for the patient
identifying the patient based on the patient identification information;
determining whether or not known support information for the identified patient is present;
generating estimated support information based on the question information in response to determination that the known support information for the identified patient is absent and
outputting the estimated support information.
